(19) United States
(12) Patent Application Publication
(10) Pub. No.: US 2005/0166239 A1
(43) Pub. Date: Jul. 28, 2005

(54) Surgery support system

(75) Inventors: Akinobu Uchikubo, Iruma-shi (JP); Takeaki Nakamura, Tokyo (JP); Masakazu Gotanda, Tsukui-gun (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 10/962,036

(22) Filed: Oct. 8, 2004

(30) Foreign Application Priority Data
Oct. 9, 2003 (JP) 2003-351222
Oct. 27, 2003 (JP) 2003-366576

(51) Int. Cl. H04N 7/173; H04N 9/47; H04N 7/18; A62B 1/04; A61B 1/04; H04N 7/025; H04N 7/10; A61B 1/00

(52) U.S. Cl. 725/78; 725/80; 725/81; 348/65; 725/111; 725/32; 600/101; 600/117; 600/921

(57) ABSTRACT

In an operation support system 1 according to the present invention, a first operation room 1A, a second operation room 1B, a third operation room 1C, . . . , a conference room 1D, and the like are connected by an IP network via an operation room communication line 8, are connected to the Internet by a WEB server 7 via an in-house communication line 6, and are externally connected via a communication line 9. A first controller 3 in the first operation room 1A has a table for registering an IP address for permitting the connection to a connecting request destination which requests the connection. A determining device 2 in the first operation room 1A determines whether the connection to the connection request destination is permitted or refused depending on the presence or absence of the IP address registered in the table when the connection request destination requests the connection.

This application claims benefit of Japanese Application Nos. 2003-351222 filed on Oct. 9, 2003, and 2003-366576 filed on Oct. 27, 2003, the contents of which are incorporated by this reference.

### BACKGROUND OF THE INVENTION

1. Field of the Invention

The present invention relates to an operation support system for connecting one operation room to another operation support room via a communication line.

2. Description of the Related Art

Conventionally, an endoscope system is widely used, in which an endoscope with an external TV camera attached to an eyepiece portion of an optical endoscope or an electronic endoscope having image pick-up means at a distal end thereof displays an endoscope image captured by the endoscope and the observation and treatment are performed while viewing the image.

The endoscope system comprises: an electronic endoscope; a light source device which supplies illumination light to the electronic endoscope; a camera controller having an image signal processing circuit for displaying the endoscope image; and a TV monitor which displays the endoscope image. Further, an aeroperitoneum device or a high-frequency cautery device is used as a peripheral device and the observation, treatment, or the operation is performed under the endoscope observation.

In the above-mentioned endoscope system, even an inexperienced operator performs the operation without fail by the observation of the endoscope image with a well-skilled operator and an instruction of the well-skilled operator. In the above endoscope system, the endoscope image of the affected part (target portion) displayed on the monitor is important. The well-skilled operator orally instructs the operator or directly instructs the operation so as to display the endoscope image of the affected part desired by the well-skilled operator, and the endoscope operation is performed smoothly.

When the well-skilled operator is at another place such as a remote place, in the endoscope system, a hospital of the operator as a supporter at the remote place is connected to an operation room of the operator who actually performs the operation via a public line. The operator in the operation room performs a proper operation with the instruction of the operation from the operator at the remote place.

The above-mentioned system for remotely supporting the operation, as well-known arts, includes an operation information display method disclosed in Japanese Unexamined Patent Application Publication No. 2000-270318 and a remote operation support system disclosed in Japanese Unexamined Patent Application Publication No. 2000-237206, which are suggested by the present applicant.

In the remote operation support system disclosed in Japanese Unexamined Patent Application Publication No. 2000-270318, the endoscope image in the operation room for the operation and a state of an operation tool are continuously observed in a remote support room by connecting the operation room to the support room via a communication line. The operation information display method disclosed in Japanese Unexamined Patent Application Publication No. 2000-270318 comprises: a step of inputting a control content of the operation tool to a controller; a step of sending the endoscope image and the control content of the operation tool to a remote place by the communication line via the controller; and a step of displaying the endoscope image and the control content of the operation tool on a monitor at the remote place.

With the above structure, the operation information display method is realized to observe the endoscope image and to support the operation such as an instruction for the operation while checking the state of the operation tool.

The remote operation support system disclosed in Japanese Unexamined Patent Application Publication No. 2000-237206 comprises: endoscope-image picking-up means which picks-up an image of the body cavity and which is arranged in the operation room; a video image sending device which is connected to a communication line and which sends, to the remote room, an image signal of the body cavity obtained by the endoscope-image pick-up means; and image signal insulating means which electrically insulates the endoscope-image pick-up means to the video image sending device and which sends an image signal.

### SUMMARY OF THE INVENTION

According to the present invention, an operation support system connects a first device arranged in an operation room to a second device arranged in an operation support room other than the operation room via a communication line and for enabling the communication to an external device via the communication line. The operation support system comprises: a setting portion which registers identification information for permitting the connection to a connecting request destination requesting the connection via the communication line; and a determining portion which determines whether the connection to the connection request destination is permitted or refused depending on the presence or absence of the identification information registered by the setting portion.

### BRIEF DESCRIPTION OF THE DRAWINGS

FIG. 1 is a block diagram showing the entire structure of an operation support system according to a first embodiment of the present invention;

FIG. 2 is a flowchart showing an example of a control operation using a determining device shown in FIG. 1;

FIG. 3 is a diagram showing the entire structure of an operation support system according to a second embodiment of the present invention;

FIG. 4 is a flowchart showing an example of a control operation using a determining device shown in FIG. 3;

FIG. 5 is a flowchart showing an example of a control operation of the partner's device which requests the connection;

FIG. 6 is a diagram showing the entire structure of an operation support system according to a third embodiment of the present invention;

FIG. 7 is a flowchart showing an example of a control operation using a determining device shown in FIG. 6;

FIG. 8 is a diagram showing the entire structure of an operation support system according to a fourth embodiment of the present invention;

FIG. 9 is a block diagram showing the structure of an operation support system and a terminal device serving as main portions shown in FIG. 8;

FIG. 10 is a block diagram showing the structure of a controller on the operation support system side;

FIG. 11 is a block diagram showing the structure of a controller on the terminal device side;

FIG. 12 is a diagram showing a monitor display example on the terminal device side;

FIG. 13 is a flowchart showing the operation sequence for setting an operation support state in automatic authenticating processing according to the fourth embodiment;

FIG. 14 is an explanatory diagram schematically showing a control operation shown in FIG. 13;

FIG. 15 is a flowchart showing the operation sequence for setting an operation support state authenticating processing according to a fifth embodiment;

FIG. 16 is a diagram showing an example of a setting screen for setting an authenticating condition in the authenticating processing; and

FIG. 17 is an explanatory diagram showing one example of a communication content upon setting the operation support states of the operation support system and a plurality of terminal devices.

### DETAILED DESCRIPTION OF THE PREFERRED EMBODIMENTS

Hereinbelow, embodiments of the present invention will be described with reference to the drawings.

### First Embodiment

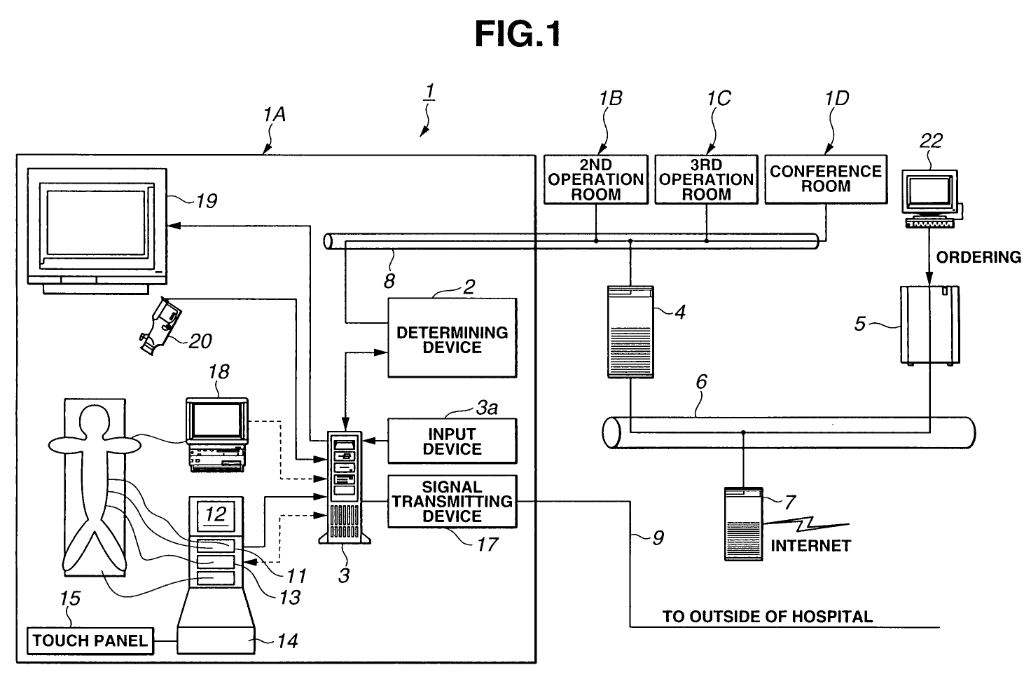

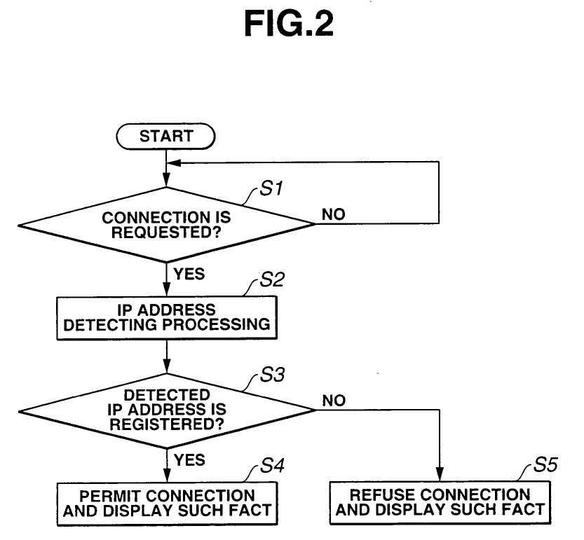

FIGS. 1 and 2 show an operation support system according to a first embodiment of the present invention. FIG. 1 is a diagram showing the entire structure of the operation support system. FIG. 2 is a flowchart showing an example of a control operation using a determining device shown in FIG. 1.

Referring to FIG. 1, an operation support system 1 according to the first embodiment comprises: a first operation room 1A in which the operation is performed under endoscope observation, serving as a supported stronghold; second and third operation rooms 1B, 1C, . . . arranged in a hospital having the first operation room 1A; a support room (hereinafter, referred to as a conference room) 1D arranged to a remote position other than the first to third operation rooms 1A to 1C; and another conference room (not shown) connected to a first controller 3 and a signal transmitting device 17 via a communication line 9. Further, the operation support system 1 comprises: the first controller 3; an operation-portion server 4; an in-house server 5; a world wide web server (hereinafter, referred to as a WEB server) 7; and a determining device 2 serving as a determining portion of the operation support system 1. The first controller 3 is a first device, serving as control means, which is arranged in the first operation room 1A, obtains various medical information generated in the operation room, determines based on a determining result of the determining device 2 whether or not the connection is permitted, and controls the sending operation of the endoscope observed image. The operation-portion server 4 is a server device which is arranged at a place other than the first operation room 1A, accumulates medical treatment information obtained by the first controller 3, and generates a medical record. The in-house server 5 is a server device which is arranged at a place other than the operation-portion server 4 and refers to medical record information obtained by the operation-portion server 4. The WEB server 7 is a WEB server device which can be connected to the Internet via an in-house communication line 6. The determining device 2 is arranged in the first operation room 1A and is connected to the first controller 3.

In the operation support system 1, the determining device 2 and the first controller 3 are connected to second devices arranged to the second operation room 1B, the third operation room 1C, . . . , and the conference room 1D and to the operation-portion server 4 via an operation-room communication line 8. The operation-portion server 4 is connected to the in-house server 5 via the in-house communication line 6. That is, in the operation support system 1, IP addresses are assigned to the devices in the first operation room 1A, the second operation room 1B, the third operation room 1C, and the conference room 1D, the operation-portion server 4, and the in-house server 5. Thus, the operation support system 1 is connected to the Intranet and the Internet via an IP network.

The communication lines 6 and 8 may be any of communication lines such as a public line, a dedicated line, and a LAN (Local Area Network). The in-house server 5 may be arranged at the same place as that of the operation-portion server 4. Although the conference room 1D is arranged in the hospital as the support room for supporting the first operation room 1A, it may be arranged at a remote place except for the hospital. In this case, the conference room 1D is connected to the IP network.

A second controller 14 connected to the first controller 3 is arranged in the first operation room 1A, and controls: an endoscope device 11 which picks-up an image of a subject such as patient's body cavity and which obtains an image signal serving as an endoscope image; a display device 12 which displays the image signal obtained by the endoscope device 11; and an operation device 13 such as an electric knife device, an aeroperitoneum device, or an ultrasonic operation device. Further, the second controller 14 obtains operation device information such as measurement information of the operation device 13.

A touch panel 15, serving as input means, which inputs a control instruction to the operation device 13, is connected to the second controller 14.

The operation device information supplied from the second controller 14 and patient information obtained from a patient monitoring device 18 are accumulated to the first controller 3 via the communication line, and are sent to the operation-portion server 4 arranged at another place via the operation-room communication line 8.

The patient monitoring device 18 connected to the first controller 3 continuously detects and monitors patient information (living-body information) such as patient's blood pressure, heartbeat, and oxygen concentration in the blood.

The first controller 3 receives the image signal obtained from the endoscope device 11 and the image signal obtained from a room camera 20 connected to the first controller 3 for picking-up an image of the situation in the operation room. Both the image signals are sent to the conference room iD via the operation-room communication line 8.

Therefore, in the operation support system 1, the conference room ID at a place other than the first operation room 1A receives and displays the image signal serving as the transmitted endoscope image and the image signal picking-up the image of the situation in the operation room onto a monitor (not shown) and thus the operator in the conference room 1D can recognize the images. Further, in the operation support system 1, the operator in the conference room 1D sends, to the first operation room 1A via the operation-room communication line 8, the image signal and the support information for support so that the operator in the first operation room 1A performs the proper operation of the patient, displays the image signal and the support information to a display device 19 which will be described later. Further, the operator in the conference room iD supports the operation by sending an instruction in real time by voice with a head set (not shown) having a microphone and an earphone.

The signal transmitting device 17 receives, via the first controller 3, the image signal obtained from the endoscope device 11 and the image signal from the room camera 20 for picking-up the image of the situation in the operation room. Further, both the image signals are sent to an external device which is externally arranged (at, e.g., another remote conference room) via the communication line 9.

The display device 19 is arranged in the first operation room 1A. The display device 19 displays information on the operation device obtained by the first controller 3, set information such as a connectable IP address, a connecting determining result of the determining device 2, patient information obtained from the operation-portion server 4, and the endoscope image signal for the operation support from the conference room 1D, and further displays information externally received by the signal transmitting device 17 via the communication line 9.

The operation-portion server 4 is connected to the in-house server 5 and the WEB server 7 via the in-house communication line 6. In the hospital, the inside of hospital is connected to the outside of hospital via the internet by the WEB server 7 and therefore necessary information from the outside of hospital can be captured.

The in-house server 5 stores the patient registered information from a patient registering terminal 22 in the hospital, an image before the operation such as an MR, CT, or X-ray image on the patient, and a previous medical image such as an image during the previous operation (e.g., endoscope moving image). The in-house server 5 receives the image signal of the endoscope image obtained from the endoscope device 11 in real time and stores the image signal.

The operation-portion server 4 reads, from the in-house server 5 via the in-house communication line 6, the medical image on the patient and registered information such as a name, birthday, and diagnostic record of the patient to be operated.

The first controller 3 performs various main control operations of the entire devices in the first operation room 1A. For example, the first controller 3 controls the storage of data such as the endoscope image and the patient information, the display operation for generating and displaying the image signal for driving the display device 19, the driving operation of the patient monitoring device 18 and the second controller 14, the sending operation for sending the living-body information and the information on the operation device via the operation-room communication line 8, the input/output operation for inputting/outputting the support information and the video information including the endoscope image to the signal transmitting device 17, the receiving/sending operation of receiving and sending the information via the operation-room communication line 8, and the connection based on the connecting determining result of the determining device 2.

Connected to the first controller 3 is an input device 3a including setting means for setting identification information for permitting the connection to the connection request destination. The input device 3a includes a keyboard, a magnetic card reader, an optical card reader, or an 1C card reader. The patient information such as medical record No. (ID No.) of the patient and name and identification information for determining whether or not the connection is permitted which will be described later are inputted by using the input device 3a.

According to the first embodiment, in a setting and registering method of the identification information, the identification information such as partner's IP address to which the connection is permitted is stored in a storing portion (not shown) in the first controller 3 by using the input device 3a before the operation by the operator, and a table for determining permission/refusal of connection is created.

In this case, the determining table is created after setting and registering data every operator or every patient. For example, a plurality of tables having parameters varied depending on each operator are provided.

The data is set to the determining table depending on respective departments in the in-house or out-house network, operators, and relating facilities, in detail. Thus, it is convenient for the operator, and the system is structured with high security.

The input device 3a may be connected to the second controller 14. When the input device 3a is connected to the second controller 14, the identification information and the patient information are sent to the first controller 3 from the second controller 14.

In the operation support system 1 according to the first embodiment, the determining device 2 connected to the first controller 3 is arranged.

Referring to FIG. 1, the determining device 2 is connected to the operation-room communication line 8 in the house, the first controller 3, and the communication line 9 externally connected (to the support room) via the signal transmitting device 17. Further, the determining device 2 is connected to the Internet via the in-house communication line 6 and the WEB server 7.

The determining device 2 determines whether or not the connection is permitted based on the identification information (setting information), which is set and registered in advance in the first controller 3 by the input device 3a, for determining whether or not the connection is permitted in response to the connection request to the first operation room 1A, and further outputs the determining result to the first controller 3.

That is, when the connection is externally requested, the determining device 2 refers to the table corresponding to the operator or patient in the first controller 3 and outputs the determining result indicating the connection refusal to the connection request except for the partner's IP address to which the connection is permitted to the first controller 3.

The first controller 3 determines whether or not the connection is permitted based on the determining result from the determining device 2, and controls the permitting or refusing operation of the connection via the communication line. That is, the first controller 3 comprises a control portion which controls the permitting or refusing operation of the connection via the communication line.

When the connection request is permitted, the first controller 3 controls the connection via the corresponding communication line, thereby sending the image signal of the endoscope image or information to the partner which requests the connection.

According to the first embodiment, the determining device 2 determines the permission or refusal of the connection. Further, the first controller 3 forcedly determines the permission or refusal of the connection based on the determining result and controls the permission and refusal of the connection via the corresponding communication line. Simultaneously with the permission or refusal, the display operation may be controlled so as to display the partner's information to which the connection is requested and the permission or refusal of connection.

According to the first embodiment, the first controller 3 may control the display operation to display the permission or refusal of connection based on the determining result from the determining device 2 on the display device 19 together with the information on the partner which requests the connection. Further, the first controller 3 controls the operation to enable the operator in the first operation room 1A to recognize the partner's information and to select the permission or refusal of connection. Therefore, the operator selects the permission or refusal of connection by operating the input device 3a, and the first controller 3 may control the operation so that the permission or refusal of connection is determined based on the selecting operation and the connection is permitted or refused via the corresponding communication line.

When the determining device 30 determines that the connection is refused, the display operation is controlled so as to display the refusal of connection on the display device 19 together with the information on the partner which requests the connection, and the operator may determine the permission or refusal of re-connection if necessary.

According to the first embodiment, the determining device 2 is arranged separately from the first controller 3. However, the present invention is not limited to this and may be integrally structured in the first controller 3 as a determining portion.

Next, a description is given of the operation of the operation support system with the above-mentioned structure with reference to FIG. 2. The identification information such as the IP address of the partner to which the connection is permitted is inputted, in advance, to the first controller 3 by using the input device 3a by the operator before the operation, and is stored, set, and registered in the table in the first controller 3.

The operation is performed in the first operation room 1A by using the operation support system shown in FIG. 1. The determining device 2 in the operation support system 1 starts and executes a processing routine shown in FIG. 2.

In determining processing in step S1, the determining device 2 determines, whether the connection is requested from any of the second operation room 1B, the third operation room 1C, the conference room 1D, and the external remote conference room. If the determining device 2 determines that the connection is not requested, the determining device 2 continues the determining processing.

On the contrary, if the determining device 2 determines in the determining processing in step S1 that the connection is requested from a third party except for the first operation room 1A, the determining device 2 advances the processing to step S2 whereupon the IP address which requests the connection is detected and the determining device 2 advances the processing to step S3.

Then, in the determining processing in step S3, the determining device 2 compares the IP address detected in step S2 with the identification information in the table in the first controller 3, and determines whether or not the detected IP address is registered.

In this case, when the determining device 2 determines that the detected IP address is registered, in step S4, the determining device 2 controls the first controller 3 so that the display device 19 displays thereon such a fact that the connection is permitted, together with the information on the partner which requests the connection and enables the operator in the first operation room 1A to recognize the fact. Then, the first controller 3 connects the operation room to the corresponding communication line, thereby sending the endoscope image or information.

Meanwhile, if it is determined in the determining processing in step S3 that the detected IP address is not registered, in the processing in step S5, the determining device 2 controls the first controller 3 so that the display device 19 displays thereon the refusal of connection together with the information on the partner which requests the connection, and enables the operator of the first operation room 1A to recognize the fact. The first controller 3 refuses the connection to the corresponding communication line, thereby disabling the transmission of the endoscope image or information.

In the processing in step S4 or S5, the display device 19 displays thereon the permitting or refusing result of the connection of the determining device 2 together with the information on the partner which requests the connection, and enables the operator in the first operation room 1A to recognize the information of the partner. Further, the first controller 3 may control the operation to enable the operator to select the permission or refusal of the connection. In this case, if the operator performs the permitting or refusing operation, the operator selects the permission or refusal by using the input device 3a. Based on the selecting operation, the first controller 3 controls the determination of connection to the corresponding communication line.

According to the first embodiment, if the connection is requested from a third party during the operation in the first operation room 1A, the permission or refusal of the connection to that except for the partner corresponding to the pre-registered IP address is determined. Thus, the troublesomeness due to the connection is removed, enabling the operator to perform the operation smoothly. Further, the endoscope image sent for support during the operation is not read by a third party which is not desired, that is, the leakage of information on the privacy is prevented and the security is improved.

### Second Embodiment

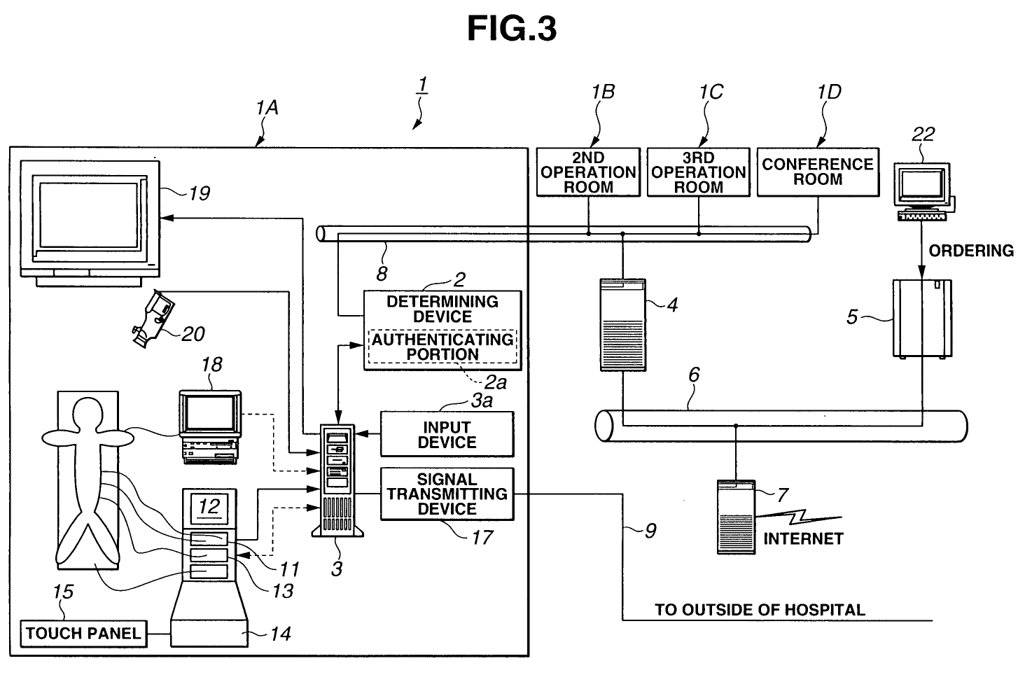

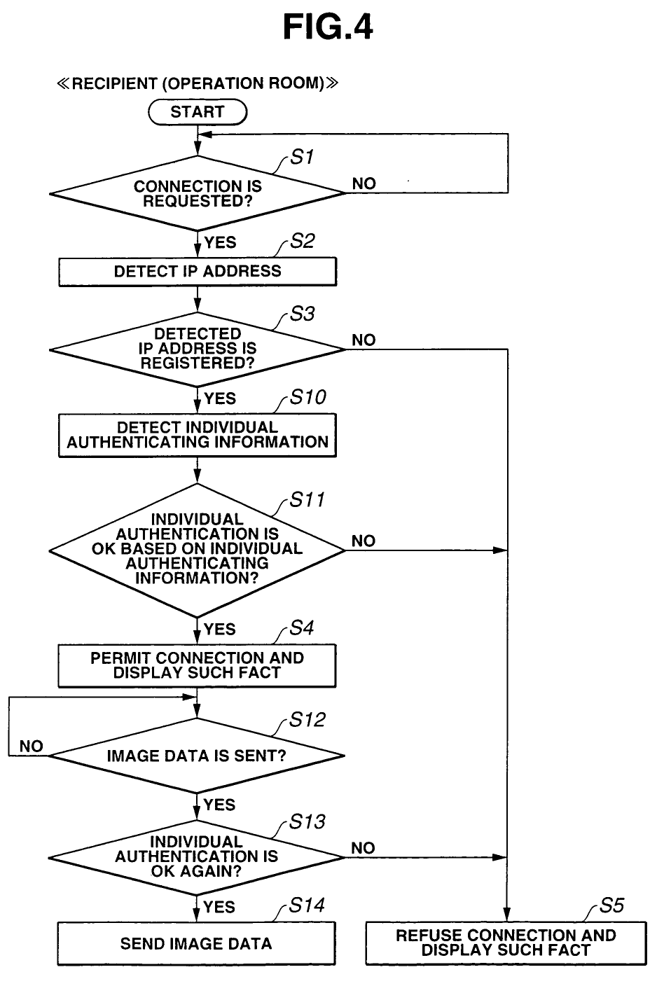

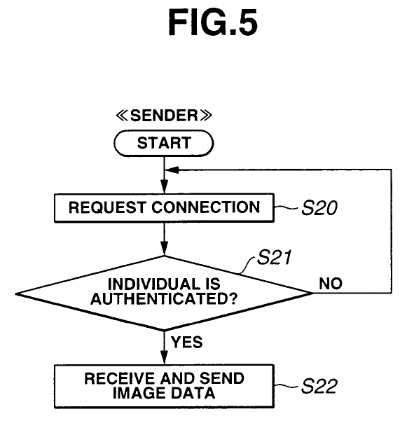

FIGS. 3 to 5 show an operation support system according to a second embodiment. FIG. 3 is a diagram showing the entire structure of the operation support system. FIG. 4 is a flowchart showing the flow of an example of a control operation of a determining device shown in FIG. 3. FIG. 5 is a flowchart showing an example of a control operation of a device of the partner which requests the connection. Referring to FIGS. 3 to 5, the same components and the same processing contents as those according to the first embodiment are designated by the same reference numerals and the same step Nos., a description thereof is omitted, and only different parts are described.

According to the second embodiment, even if the connection is permitted in response to the connecting request from a third party and then the image signal as the endoscope image or the individual data such as the patient information is sent, the individual is authenticated.

Referring to FIG. 3, the operation support system 1 comprises an authenticating portion 2a for authenticating the individual in the determining device 2.

When the determining device 2 permits the connecting permission of the connecting request and the image signal of the endoscope image or the individual data such as the patient information is sent, the authenticating portion 2a determines the permission or refusal of the sending operation of the sender. For example, the authenticating portion 2a detects individual authenticating information (living-body information) such as the fingerprint or retina of the sender, and compares the detected information with the individual authenticating information which is registered for the sending permission in advance, thereby determining the permission or refusal.

The authenticating portion 2a comprises an existing fingerprint authenticating device or an existing retina authenticating device. However, the present invention is not limited to this and the authenticating portion 2a may be another device which can authenticate the individual. Similarly to the first embodiment, it is necessary to pre-register the individual authenticating information of the sender corresponding to the sending permission necessary for authenticating the individual. Thus, the determining device 2 or the authenticating portion 2a comprises a storing portion (not shown, hereinafter, referred to an individual authenticating table) which sets and registers in advance the individual authenticating information to the individual authenticating table. In this case, the individual authenticating table may be arranged in the first controller 3. In this case, the authenticating portion 2a authenticates the individual by referring to the individual authenticating table (not shown) in the first controller 3.

The determining result of the authenticating portion 2a is outputted to the first controller 3. The first controller 3 controls the sending operation of the image signal as the endoscope image or image signal by the sender to be executed or be prohibited.

Other structures are the same as those according to the first embodiment.

According to the second embodiment, the authenticating portion 2a authenticates the individual of the sender in the first operation room 1A. However, the individual serving as the sending destination of the partner who requests the connection may be authenticated. In this case, the individual authenticating information (e.g., name or ID) for sending the image signal as the endoscope image or individual data is registered in advance in another individual authenticating table arranged to the determining device 2 or the authenticating portion 2a. Further, the individual authenticating information of the sending destination is inputted by using the input device 3a, thereby authenticating the individual by the authenticating portion 2a.

Next, a description is given of the operation of the operation support system 1 according to the second embodiment with reference to FIGS. 4 and 5.

When the operation is performed in the first operation room 1A by using the operation support system shown in FIG. 3, the determining device 2 starts to execute a processing routine shown in FIG. 4.

In the determining processing in step S1, the determining device 2 determines, whether the connection is requested from any of the second operation room 1B, the third operation room 1C, the conference room 1D, and the external remote conference room. If the determining device 2 determines that the connection is not requested, the determining device 2 continues the determining processing.

On the contrary, if the determining device 2 determines in the determining processing in step S1 that the connection is requested from a third party except for the first operation room 1A, the determining device 2 advances the processing to step S2 whereupon the IP address from which the connection is requested is detected and the determining device 2 advances the processing to step S3.

Then, in the determining processing in step S3, the determining device 2 compares the IP address detected in step S2 with the identification information in the table in the first controller 3, and determines whether or not the detected IP address is registered.

In this case, when the determining device 2 determines that the detected IP address is detected, the determining device 2 advances to step S10. If the determining device 2 determines that the detected IP address is not detected, the determining device 2 advances to step S5 whereupon the first controller 3 controls the operation so that the display device 19 displays thereon the refusal of connection together with the information of the partner which requests the connection and enables the operator to recognize the fact. Further, the first controller 3 refuses the connection to the corresponding communication line, and thus the image signal as the endoscope image or information cannot be sent.

In the processing, similarly to the first embodiment, the display device 19 displays thereon the refusal of connection of the determining device 2 together with the information of the partner which requests the connection, and enables the operator in the first operation room 1A to recognize the information of the partner. Further, the first controller 3 may control the operation so that the operator select the permission or refusal of connection if necessary.

In the processing in step S10, the determining device 2 detects the individual authenticating information by the authenticating portion 2a. In the processing in step S11, the determining device 2 compares the detected individual authenticating information with the individual authenticating information (living-body information) which is set and registered in advance in the individual authenticating table (not shown) in the determining device 2 or authenticating portion 2a, thereby authenticating the individual.

In this case, if the individual is not authenticated, the determining device 2 advances the processing to step S5 whereupon the above-mentioned processing is executed. If the individual is authenticated (OK), in the processing in step S4, the first controller 3 is controlled so that the display device 19 displays thereon the permission of connection together with the information of the partner which requests the connection, and enables the operator in the first operation room 1A to recognize the information of the partner. The first controller 3 performs the connection to the corresponding communication line, thereby sending the image signal as the endoscope image or the information.

In the processing, similarly to the first embodiment, the display device 19 displays thereon the permission of connection using the determining device 2 together with the information of the partner which requests the connection and enables the operator in the first operation room 1A to recognize the information of the partner. Further, the first controller 3 may control the operation so that the operator selects the permission or refusal of connection if necessary.

It is assumed that the operator in the first operation room 1A sends the image signal of the endoscope image or individual data to the partner to which the connection is permitted.

The determining device 2 shifts to the processing in step S12 whereupon it determines whether or not the image signal of the endoscope image or individual data is sent. If the determining device 2 determines that the image signal or individual data is not sent, the determining device 2 continues the determining processing. If the determining device 2 determines that the image signal or individual data is sent, the determining device 2 advances to the processing to step S13.

In the determining processing in step S13, the determining device 2 authenticates the individual again, similarly to step S11. That is, based on the determining result of the individual authentication, the individual is authenticated again to determine whether or not the individual is a concerned one so as to send the image signal of the endoscope image or individual data with the privacy.

If the individual is not authenticated, in step S5, the determining device 2 performs the similar processing. While, on the other hand, if the individual is authenticated (OK), in the processing in step S14, the determining device 2 issues a control instruction to the first controller 3, and sends the image signal of the endoscope image or individual data to the partner who requests the connection via the communication line.

FIG. 5 shows a control example of the device of the partner who requests the connection.

According to the second embodiment, in the processing in step S20, a control portion for controlling the partner's device requests the connection to the first operation room 1A. After that, in step S21, the control portion determines whether or not the individual is authenticated on the first operation room 1A side. If it is determined that the individual is not authenticated, the processing returns to step S20 whereupon the connection is requested again. If it is determined that the individual is authenticated, in the processing in step S22, the control portion controls the operation to receive the image signal of the endoscope image sent from the first operation room 1A or to send the information such as the instruction or the image signal of the endoscope image for support from/to a receiving/sending portion of the control device.

The control device for the above operation is arranged not only to the second operation room 1B, the third operation room 1C, the conference room 1D, and the remote support room but also to another relating facility, the system having the higher security can be structured.

Thus, according to the second embodiment, the same advantages as those according to the first embodiment are obtained. Further, if the connection is permitted in response to the connecting request from a third party, the higher security can be ensured by authenticating the individual upon sending the image signal of the endoscope image or the individual data such as the patient information.

### Third Embodiment

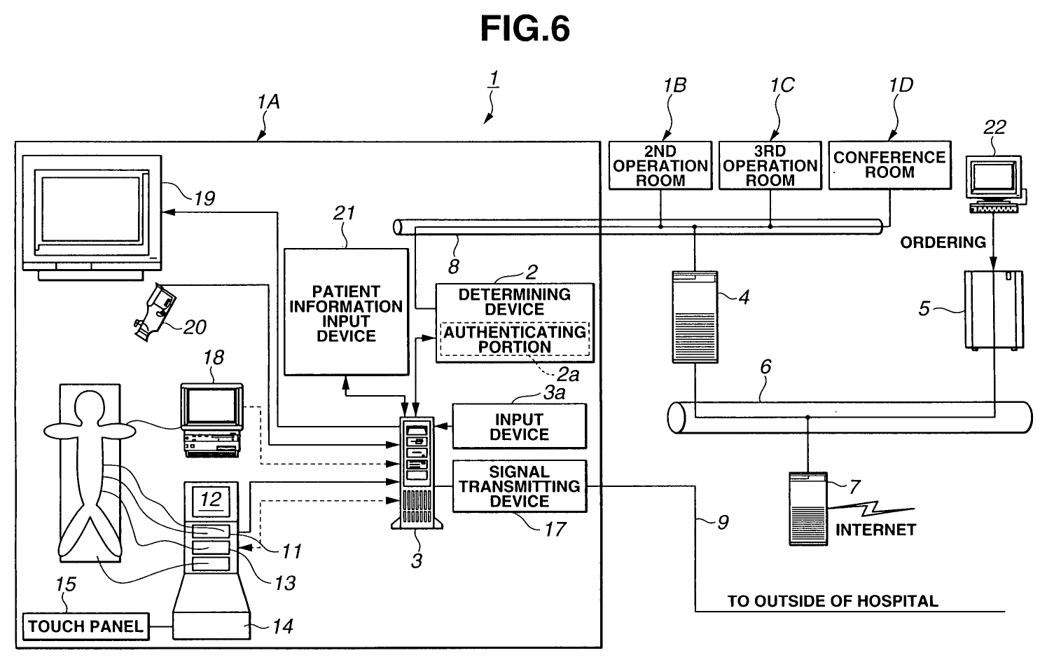

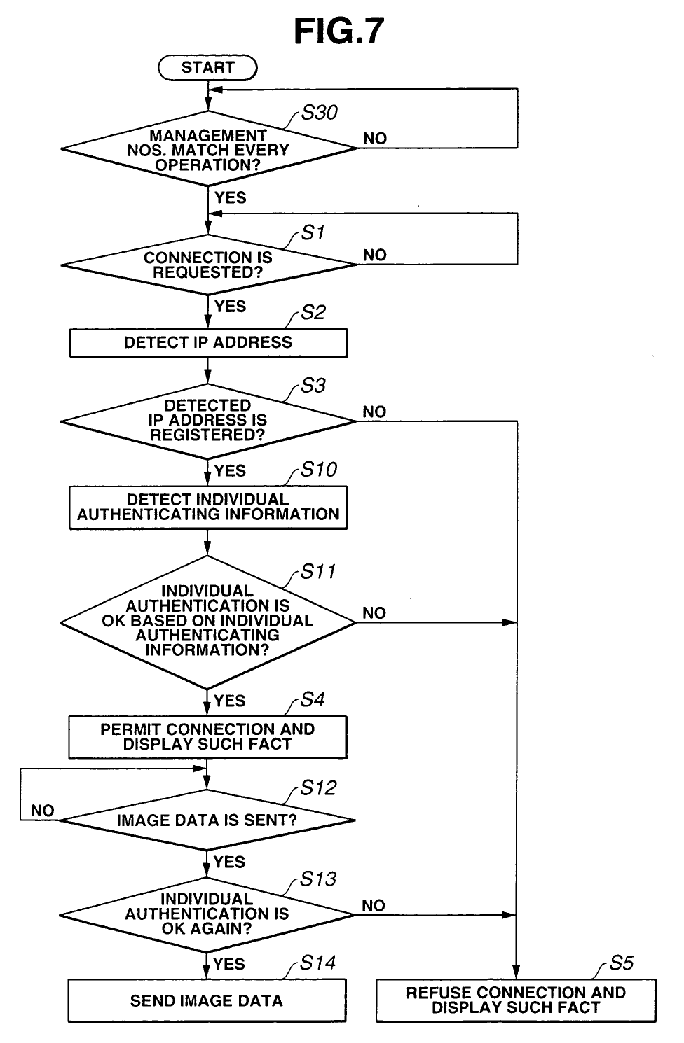

FIGS. 6 and 7 show an operation support system according to a third embodiment of the present invention. FIG. 6 is a diagram showing the entire structure of the operation support system. FIG. 7 is a flowchart showing an example of a control operation using a determining device shown in FIG. 6. Referring to FIGS. 6 and 7, the same components and the processing as those according to the first and second embodiments are designated by the same reference numeral and the same step No., a description thereof is omitted, and only different portions are described.

According to the third embodiment, there is a feature that the determining processing of the permission or refusal in response to the connecting request from a third party by using the determining device 2 is executed every operation in the first operation room 1A.

Referring to FIG. 6, the operation support system 1 according to the third embodiment comprises a patient information input device 21 for capturing the patient information such as management No. for executing the determining processing of the permission or refusal in response to the connecting request from a third party by using the determining device 2 is executed every operation. The patient information input device 21 is connected to the first controller 3.

The patient information input device 21 captures the patient information for identifying the operation in the first operation room 1A via input means such as a keyboard (not shown), and supplies the captured information to the determining device 2 via the first controller 3.

The patient information is a management No. such as a schedule No. of the operation determined every operation, patient ID for the operation, or ID of the operator who performs the operation. Further, the patient information may be another for identifying the single operation.

The patient information is captured in advance in the first controller 3 by the patient information input device 21. Further, the patient information is set and registered in the table for determining the permission/refusal of connection which registers the setting information such as the IP address of the partner whose connection is permitted, in the first controller 3.

When the connection is requested, the determining device 2 compares the patient information inputted by the patient information input device 21 with the patient information in the table which is previously set and registered before determining the permission or refusal of connection in response to the connecting request, and determines the permission or refusal of connection in response to the connecting request only when both the patient information matches each other.

Other structures are substantially the same as those according to the second embodiment.

Next, a description is given of the operation of the operation support system with reference to FIG. 7 according to the third embodiment.

In the operation support system 1 according to the third embodiment, upon using the operation support system 1, the determining device 2 starts to execute a processing routine shown in FIG. 7.

The determining device 2 executes the determining processing in step S30. In the determining processing, the determining device 2 compares the patient information (e.g., management No.) inputted by the patient information input device 21 with the patient information (management No.) in the previously-set and registered table and thus determines whether or not both the patient information matches each other.

When the determining device 2 determines that both the patient information does not match each other, the determining device 2 continues the determined processing. When the determining device 2 determines that both the patient information matches each other, the determining device 2 executes the same processing routine (steps S1 to S14) as those according to the second embodiment so as to determine the permission or refusal of the requested connection.

Steps S1 to S14 are the same as those according to the second embodiment and therefore a description thereof is omitted.

According to the third embodiment, in addition to the advantages according to the second embodiment, the determining processing of the permission or refusal of the connection requested from a third party by the determining device 2 is executed every operation in the first operation room 1A. Thus, the operation support system with the higher security is structured.

According to the first to third embodiments, in the determining processing of the permission or refusal of the connection using the determining device 2, the IP address as the identification information is used. However, the present invention is not limited to this and any of the tel. No. of a dial-up network and the IP address may be used for the control operation as the identification information.

Further, in the operation support system according to the first to third embodiments, the permission or refusal of the connection is determined mainly for the first operation room 1A and the connection is determined based on the determining result. However, the present invention is not limited to this and the determining processing of the permission or refusal of connection requested is performed mainly for the conference room 1D or the external conference room (not shown) connected by the communication line 9.

In this case, the conference room 1D or the external conference room comprises at least a table for storing the identification information for determining the connection. Further, the conference room 1D or the external conference room comprises: a controller which displays, on the monitor, the image signal of the endoscope image or the information sent via the operation-portion server 4 and which controls the determination of the corresponding communication line based on the determining result of the permission or refusal; a determining device which determines the permission or refusal of the requested connection based on the identification information stored in the table; and an input device which inputs the identification information and sets and registers in the table. With the above structure, the same advantages as those in the operation support system using the first operation room 1A as the main are obtained.

According to the related arts, when a third party in the room except for the operation room knows the information such as the IP address of the network or tel. No. of the dial-up network in the operation room, the current operation is accessed and thus the connection damages the operation of the operator or causes a danger of the leakage of the information of the privacy.

In the above-mentioned conventional remote operation support system, the endoscope image is sent between the operation room and the remote position so as to support the operation of the operator in the operation room by the operator at the remote position by using the endoscope observed image. During the operation in the operation room, the operation room is accessed from another operation room in the hospital connected to the network or external position, in addition to the support room at the remote position connected to the operation room by the communication line.

That is, when a third party in the room except for the operation room knows the information such as the IP address of the network or tel. No. of the dial-up network in the operation room, the operation room during the operation is accessed and thus the connection causes the trouble of the operation or the endoscope image sent during the operation for support is read. Thus, the information on the privacy may be leaked and thus it is necessary to sufficiently ensure the security.

In the conventional operation support system disclosed in Japanese Unexamined Patent Application Publication No. 2000-270318 or Japanese Unexamined Patent Application Publication No. 2000-237206, the means for safely or efficiently performing the operation for the operation room or the operator is disclosed, however, the prevention of a disturbance in the operation of the operator due to the connection to the operation room during the operation and the improvement in security are not disclosed.

On the contrary, in the system according to the first to third embodiments, it is possible to determine the connection to the operation room from the external request based on the identification information which is set in advance. Thus, it is possible to realize the operation support system in which the troublesomeness of the operation due to the connection is removed and the security performance is improved.

The operation support system according to the first to third embodiments may comprise a controller including the determining device which determines the connection to the operation room from the external request based on the identification information which is previously set at a plurality of other operation rooms in or out of the hospital or another conference room within the network.

### Forth Embodiment

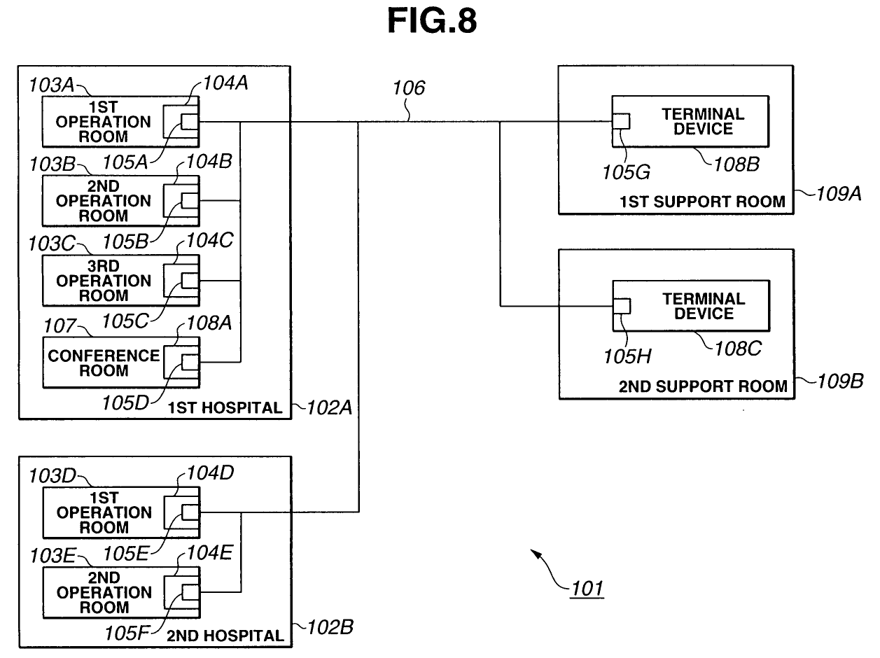

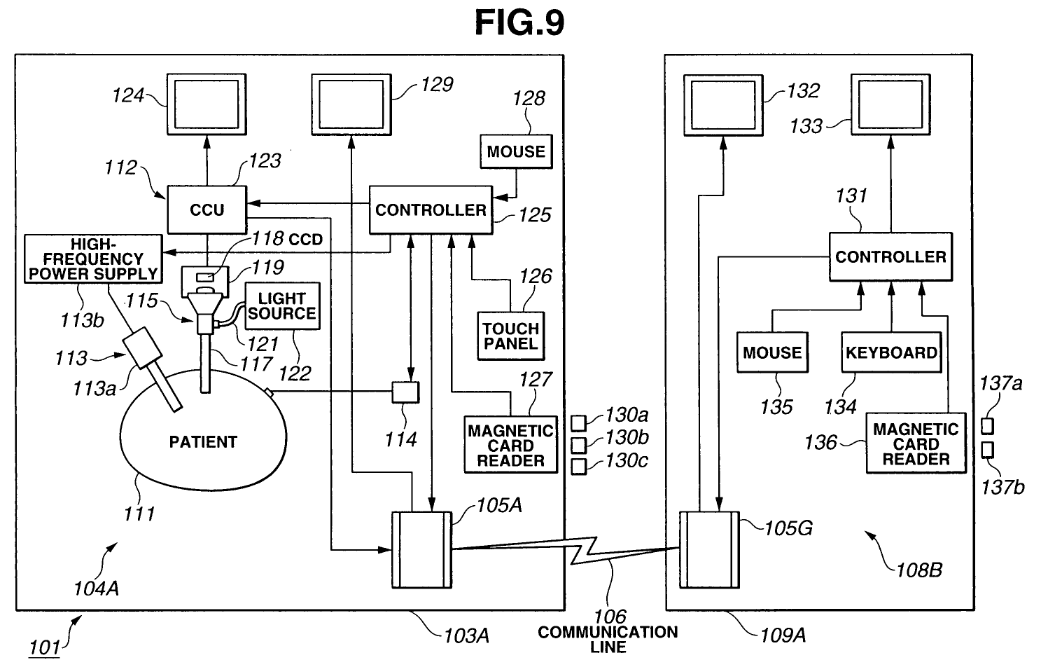

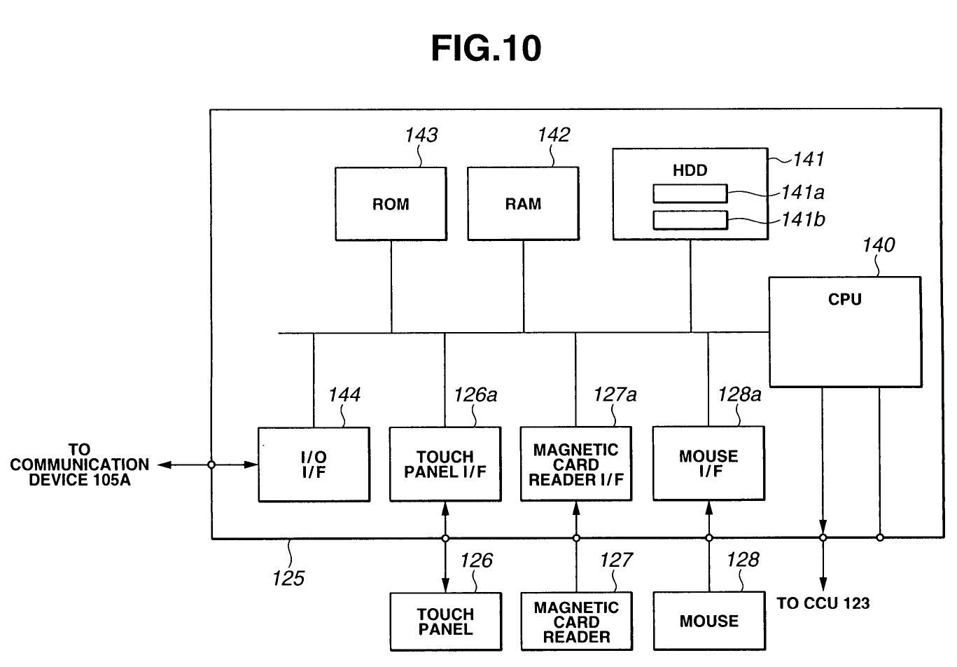

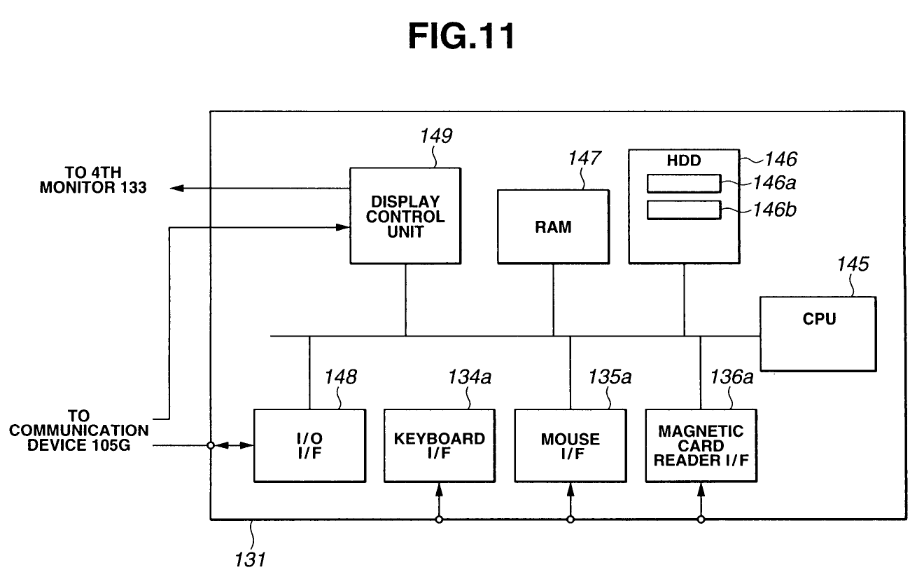

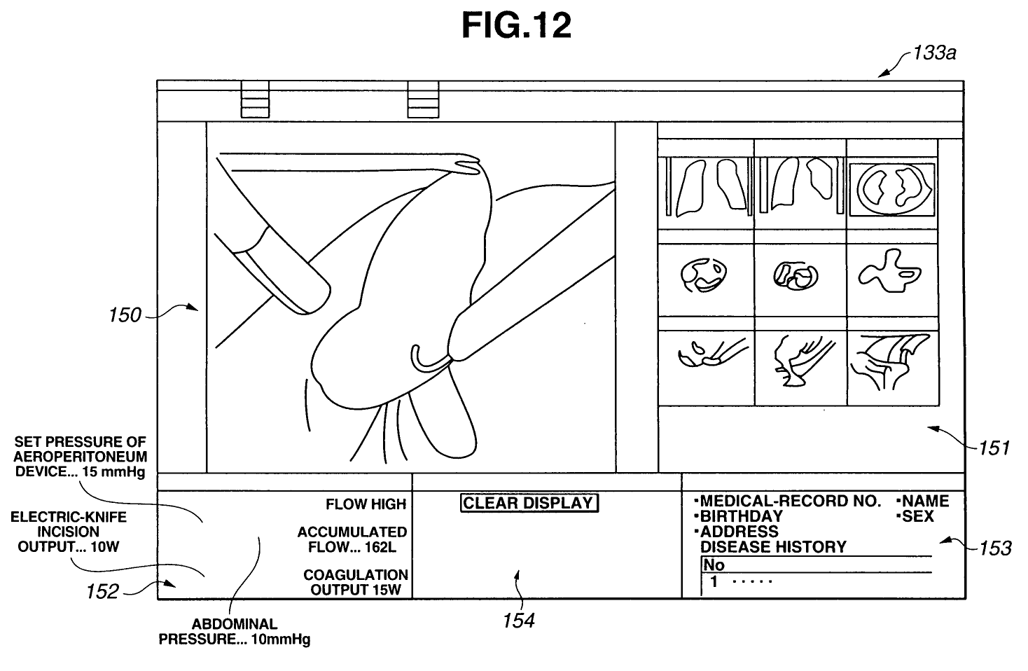

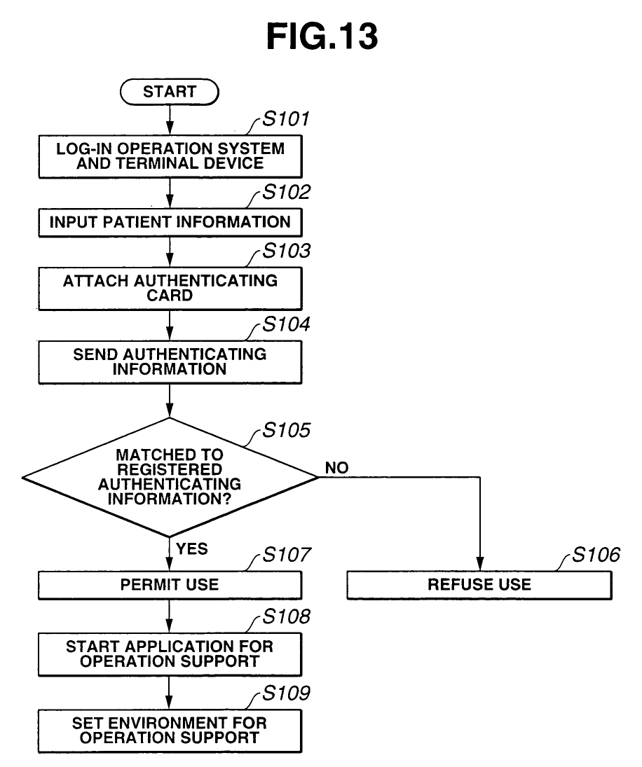

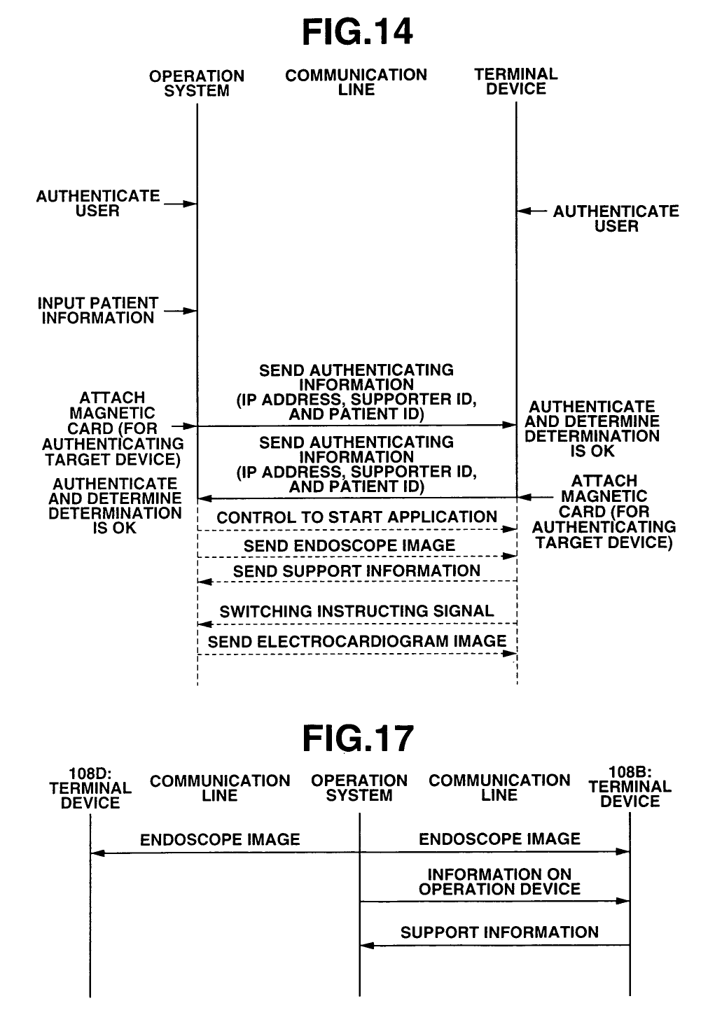

FIGS. 8 to 15 show the fourth embodiment of the present invention. FIG. 8 shows the entire structure of a remote operation support system according to a fourth embodiment. FIG. 9 shows the structure of a main portion shown in FIG. 8. FIG. 10 shows the structure of a controller on the operation system side. FIG. 11 shows the structure of a controller on the terminal device side. FIG. 12 shows a monitor display example on the terminal device side. FIG. 13 shows an operating sequence for setting the operation support state by the automatic authenticating processing according to the fourth embodiment. FIG. 14 schematically shows a control operation shown in FIG. 13.

According to the fourth embodiment, a remote operation support system for automatic authenticating processing via the communication line is provided so as to establish the communication of information for supporting the operation via the communication line between the operation room and the support room at the remote position.

Referring to FIG. 8, a remote operation support system 101 according to the fourth embodiment comprises operation systems 104A, 104B, and 104C arranged in a first operation room 103A, a second operation room 103B, and a third operation room 103C in a first hospital 102A. The operation systems 104A, 104B, and 104C are connected to a communication line 106 via communication devices 105A, 105B, and 105C.

A terminal device 108A arranged to a conference room 107 in the first hospital 102A is connected to the communication line 106 via a communication device 105D.

Operation systems 104D and 104E arranged in a first operation room 103D and a second operation room 103E in a second hospital 102B are connected to the communication line 106 via communication devices 105E and 105F.

A terminal device 108B arranged to a first support room 109A remote from the first hospital 102A or the second hospital 102B is connected to the communication line 106 via a communication device 105G.

A terminal device 108C arranged to a second support room 109B remote from the first hospital 102A and the like is connected to the communication line 106 via a communication device 105H.

An operation system 104I arranged in an operation room 103I (I=A to E) needs to prevent the leakage of information on privacy such as the patient information to the outside of the operation room 103I via the communication line 106 connected to the operation system 104I because of the operation on the patient using information relating to the operation such as patient data and endoscope images of the affected part of the patient.

According to the fourth embodiment, in order to set the communication state between the operation system 104I and a terminal device 108J (J=A to C, hereinafter, similar) via the communication line 106, a condition of predetermined authenticating processing needs to be satisfied, which will be described later. Thus, it is possible to prevent the leakage of information of the privacy.

Next, a description is given of the structure of the operation system 104A in the first operation room 103A and the structure of the terminal device 108B in the first support room 109A with reference to FIG. 9.

The operation system 104A arranged in the first operation room 103A comprises: an endoscope image pick-up device 112 which observes the body cavity of a patient 111; an electric knife device 113, serving as an operation device, which performs the operation for treatment of the patient 111 under the observation of the endoscope image pick-up device 112; an electrocardiogram measurement device 114 which measures the electrocardiogram of the patient 111; and an aeroperitoneum device (not shown).

An electric knife 113a, serving as an operation tool main body, for the operation of the treatment and an optical endoscope 115 for observing the operation state of the electric knife 113a are inserted in the abdominal part of the patient 111.

The electric knife 113a is connected to a high-frequency power supply device 113b having a function for supplying driving power to the electric knife 113a and for changing the setting of an output value in accordance with an incision or coagulation mode. The electric knife device 113 comprises the electric knife 113a and the high-frequency power supply device 113b.

An endoscope 115 is a rigid endoscope having a rigid inserting portion 117. A TV camera 119 including a charge coupled-device (hereinafter, abbreviated to a CCD) 118 serving as an image pick-up element is attached to an eyepiece portion arranged to the rear end of the inserting portion 117. Image pick-up means for picking-up the endoscope image comprises the CCD 118.

A light guide cable 121 of the endoscope 115 is connected to a light source device 122. Illumination light of a lamp (not shown) in the light source device 122 is transmitted via a light guide in the light guide cable 121 and a light guide in the endoscope 115. Further, the illumination light sent from a light-guide distal end surface fixed to an illuminating window on the distal end side of the inserting portion 117 is irradiated and the irradiated illumination light illuminates the subject side of the organ in the body cavity.

An objective lens (not shown) is attached to an observing window adjacent to the illuminating window, and an optical image of the subject is formed via the objective lens. The optical image is sent to the rear side by a relay lens system serving as optical image sending means arranged in the inserting portion 17.

The TV camera 119 is detachably attached to the eyepiece portion, thereby forming the optical image of the subject on the CCD 118 via an image forming lens inside thereof. The CCD 118 is connected to a camera control unit (hereinafter, abbreviated to a CCU) 123 via a signal cable. The CCU 123 processes the signal photoelectrically-converted by the CCD 118 and generates a video signal.

The video signal is outputted to a first monitor 124 from the CCU 123. The first monitor 124 displays thereon the endoscope image of the organ in the body cavity picked-up by the CCD 118 and of the distal end side of the electric knife 113a for operating the organ in the body cavity.

The endoscope image pick-up device 112 comprises: the endoscope 115; the TV camera 119; the CCU 123; and the first monitor 124 and the like.

The CCU 123, the high-frequency power supply device 113b, and the electrocardiogram measurement device 114 are connected to a (first) controller 125 for controlling them.

Connected to the controller 125 as a first device are a touch panel 126 for inputting a control instruction, a magnetic card reader 127 for inputting the patient data or the ID information of an operator, and a mouse 128 and the like. In place of the magnetic card reader 127, an 1C card reader or the like may be connected to the controller 125.

The operator controls the operation for changing the tone by using the CCU 123 via the controller 125 by operating the touch panel 126, and further controls the output of the electric knife 113a via the high-frequency power supply device 113b. Furthermore, the operator controls the setting of the output level suitable to the incision and coagulation using the electric knife 113a by operating the touch panel 126. In addition, the operator sets and varies a value of a set pressure of the aeroperitoneum device (not shown) by operating the touch panel 126.

The user attaches a magnetic card 130a for inputting the patient information to the magnetic card reader 127. Thus, the magnetic card reader 127 reads the patient data recorded to the magnetic card 130a, and inputs the patient data to the controller 125.

The controller 125 records the inputted patient data to recording means therein, and outputs the patient data to the CCU 123. The user selects the operation for imposing and displaying the patient data and, then, the CCU 123 imposes the patient data to the generated endoscope image, outputs the imposed data to the first monitor 124, and displays the patient data on the monitor screen.

The operator attaches a magnetic card 130b to which the ID information of the operator is written to the magnetic card reader 127, thereby performing the processing for authenticating the user so as to set the operation system 104A to be used.

That is, the magnetic card 130b is attached to the magnetic card reader 127, then, a user name, password, and the ID information of the operator which are recorded to the magnetic card 130b are read, and the ID information is sent to the controller 125. The controller 125 determines whether or not the ID information matches the ID information which is registered in advances in the controller 125. If the controller 125 determines that the ID information matches each other, the controller 125 determines that the user authenticating processing is normally performed and sets the operation system 104A to be used.

The controller 125 is connected to the communication device 105A connected to the communication line 106. The communication device 105A includes a communication interface and a signal converting circuit. After the authenticating processing for an external device of the first operation room 103A via the communication line 106 under the control of the controller 125, the endoscope image data or patient data generated in the CCU 123 becomes enabled to send to the external device.

In this state, the communication device 105A has a function for receiving the information sent from the external device under the control of the controller 125, sending the received information to the controller 125, further sending the received image information to a second monitor 129, and displaying the information on the monitor screen.

Meanwhile, a terminal device 108B in the first support room 109A connected to the communication line 106 comprises: a communication device 105G connected to the communication line 106; a (second) controller 131 and a third monitor 132 connected to the communication device 105G; a fourth monitor 133 serving as display means connected to the controller 131; and a keyboard 134, a mouse 135, and a magnetic card reader 136 which are connected to the controller 131.

The supporter inputs or selects the support information by operating the keyboard 134 or the mouse 135.

Similarly to the communication device 105A, the communication device 105G includes a communication interface (I/F) and a signal converting circuit. Further, the communication device 105G is connected, via the Internet, to an external device of the first support room 109A connected to the communication line 106 via the communication line 106 under the control of the controller 131.

The supporter attaches a magnetic card 137a for supporter to which the ID information of the supporter is written to the magnetic card reader 136, thereby performing the user authenticating processing for setting the terminal device 108B to be used.

That is, the magnetic card 137a is attached to the magnetic card reader 136 and, then, the magnetic card reader 136 reads the user name, password, and the ID information of the supporter recorded to the magnetic card 130b and sends the ID information to the controller 131.

The controller 131 as a second device determines whether or not the ID information matches the ID information which is registered in advances in the controller 131. If the controller 131 determines that the ID information matches each other, the controller 131 determines that the user authenticating processing is normally performed and sets the terminal device 108B to be used.

According to the fourth embodiment, the operator performs the authenticating processing of the terminal device 108B via the communication line 106 from the operation system 104A. The supporter performs the authenticating processing of the operation system 104A via the communication line 106 from the terminal device terminal device 108B. Thus, the operation system 104A and the terminal device 108B can be set to be used via the communication line 106.

In this case, in order to improve the convenience, according to the fourth embodiment, the authenticating processing is automatically performed as follows.

The operator attaches an authenticating magnetic card 130c for the terminal device 108B as the target used by the supporter for the operation to the magnetic card reader 127, thereby automatically performing the authenticating processing (authenticating procedure) of the terminal device 108B as the target having a necessary condition of the authenticating processing.

Similarly, on the terminal device 108B side, the supporter attaches an authenticating magnetic card 137b of the operation system 104A as the support target used by the operator to the magnetic card reader 136, thereby automatically performing the authenticating processing of the operation system 104A as the target having a necessary condition of the authenticating processing via the communication line 106.

Other operation systems 104B to 104E shown in FIG. 8 basically have the same structure as that of the operation system 104A. Other terminal devices 108A and 108C basically have the same structure as that of the terminal device 108B.

FIG. 10 shows the structure of the controller 125.

The controller 125 comprises: a central processing unit (hereinafter, abbreviated to a CPU) 140 for control operation; a hard disk (hereinafter, abbreviated to an HDD) 141 which stores an operation program and an image of the CPU 140; a RAM 142 used for temporary storage of the image or a working area; a ROM 143 to which the information on the operation program is written; an input/output interface (abbreviated I/O I/F in FIG. 10) 144 which inputs/outputs the control signal via the communication device 105A; and a touch panel I/F 126a, a magnetic card reader I/F 127a, and a mouse I/F 128a which are connected to the touch panel 126, the magnetic card reader 127, and the mouse 128.

The CPU 140 is connected to the CCU 123, the high-frequency power supply device 113b, and the electrocardiogram measurement device 114 via a port, thereby controlling the CCU 123, the high-frequency power supply device 113b, and the electrocardiogram measurement device 114.

Further, the CPU 140 controls the communication device 105A connected via the input/output I/F 144, and sends the image data and the like or receives the support information via the communication device 105A.

For example, the operator inputs character information by operating the touch panel 126, and displays the character information inputted on the touch panel 126. Further, the operator operates a send key arranged to the touch panel 126 and thus the CPU 140 sends the displayed character information via the communication device 105A. As means for inputting the character information, a keyboard may be arranged.

Furthermore, the CPU 140 controls the units of the controller 125 and the entire devices forming the operation system 104A connected to the controller 125. Further, the CPU 140 controls the operations in accordance with program data stored in a program storing area 141a of the HDD 141. The program data stored in the program storing area 141a includes program data for the authenticating processing for automatically performing the authenticating procedure (authenticating processing).

A program storing area 141b of the HDD 141 stores authenticating information (registered information) which is necessary for determining whether the connection is permitted or refused in the authenticating processing, e.g., the IP address of the device (terminal device 108B in FIG. 8) as the target for the operation with the operation support, the ID information of the supporter, and the patient ID.

FIG. 11 shows the structure of the controller 131 forming the terminal device 108B.

The controller 131 comprises: a CPU 145 for the control operation; a hard disk drive (hereinafter, abbreviated to an HDD) 146 which stores the operation program and the image data of the CPU 145; a RAM 147 which is used for temporary storage of the image or working area; and an input/output I/F 148 which inputs/outputs the image data or the control signal via the communication device 105G.

The controller 131 further comprises: a display control portion 149 which generates a video signal displayed on the fourth monitor 133, controls the operation for capturing the inputted video signal, and performs video processing for overlay display operation; and a keyboard I/F 134a, a mouse I/F 135a, and a magnetic card reader I/F 136a which are connected to the keyboard 134, the mouse 135, and the magnetic card reader 136, respectively. The CPU 145 is connected to the HDD 146 via a bus.

The CPU 145 is connected to the communication device 105G via the input/output I/F, and the CPU 145 controls the operation of the communication device 105G.

The CPU 145 performs the control operation of the controller 131 in accordance with the program data stored in a program storing area 146a in the HDD 146. A program storing area 146a in the HDD 146 stores program data of application for operation support and program data for the authenticating processing for automatically performing the authenticating processing.

A program storing area 146b in the HDD 146 stores authenticating information (registered information) which is necessary for determining whether the connection is permitted or refused in the authenticating processing, e.g., the IP address of the device (operation system 104A in FIG. 8) as the target for the operation with the operation support, the ID information of the operator, and the patient ID.

A display control portion 149 generates the video signal which is displayed on the fourth monitor 133 under the control of the CPU 145, captures the received video signal, stores the captured video signal into the image storing area of the HDD 146, outputs the video signal on the fourth monitor 133, and displays the video signal.

The video signal generated by the display control portion 149 is sent to the input/output I/F 148 and the communication device 105G. The image stored in the HDD 146 is selected by the keyboard 134 or the like and thus the CPU 145 outputs a reduced image (thumbnail image) of the selected image to the display control portion 149 side and imposes the thumbnail image to the captured video signal (video signal from the communication device 105G) in the display control portion 149.

FIG. 12 shows a display example on the fourth monitor 133. In the display example, the authenticating processing is normally performed between the operation system 104A and the communication device 105G via the communication line 106 and the communication of the information for operation support is possible.

A display screen 133a of the fourth monitor 133 comprises: an endoscope image display area 150; a thumbnail display area 151; an operation-tool state display area 152; a patient information display area 153; and a comment display area 154.

The endoscope image display area 150 displays a still image of the endoscope image which is captured image from the CCU 123 forming the endoscope image pick-up means, and the thumbnail display area 151 adjacent to the endoscope image display area 150 displays the selected thumbnail image.

The thumbnail display area 151 displays the reduced image (thumbnail image) of the image data stored in the HDD 146 of the controller 131.

The HDD 146 stores the still image of the endoscope image captured via the display control portion 149 from the CCU 123 in the first operation room 103A.

The operation-tool state display area 152 displays the setting state of the operation device sent from the controller 125.

The patient information display area 153 displays the patient information from the controller 125. The comment display area 154 adjacent to the patient information display area 153 displays the character information sent from the first operation room 103A.

A description is given of the operation of the remote operation support system 101 with the above-mentioned structure.

Briefly, a description is given of the operation for setting the using state for the operation support via the communication line 106 between the operation system 104A in the first operation room 103A and the terminal device 108B in the first support room 109A. The operation in this case is schematically shown in FIGS. 13 and 14.

Referring to FIG. 13, in step S101, the operator in the first operation room 103A turns on the power of the operation system 104A, and the supporter in the first support room 109A turns on the power of the terminal device 108B.

Then, the operator and the supporter attach the magnetic cards 130b and 137b to which the user names, the passwords, and the ID information are written to the magnetic card readers 127 and 136. Thus, the user names, the passwords, and the ID information of the operator and the supporter are read.

If the read information matches the ID information for authenticating the user which is registered in the HDDs 141 and 146 in the operation system 104A and the terminal device 108B, the user authentication is successful and then the user authentication normally ends. The operation system 104A and the terminal device 108B can be operated and receive the data by the users serving as the operator and the supporter.

In step S102, the operator attaches the magnetic card 130a for inputting the patient information to the magnetic card reader 127 and thus the magnetic card reader 127 reads the patient information of the magnetic card 130a and inputs the read information to the controller 125. By inputting the patient information, the operation system 104A can be used by the operator when the operation support is not necessary for the patient.

In step S103, the operator attaches the authenticating magnetic card 130c of the terminal device 108B as the (operation support) target to the magnetic card reader 127. The supporter attaches the authenticating magnetic card 137b of the operation system 104A as the target to the magnetic card reader 136.

The controllers 125 and 131 obtain the authenticating information necessary for authenticating such as the IP addresses, the user IDs, and the patient IDs of the terminal device 108B and the operation system 104A as the connection destinations from the authenticating magnetic cards 130c and 137b attached to the magnetic card readers 127 and 136.

In step S104, the controllers 125 and 131 send the obtained authenticating information to the terminal device 108B and the operation system 104A as the connecting destinations. In this case, encrypting means may be arranged so that the data is encrypted and is sent.

In step S105, the CPU 145 of the controller 131 in the terminal device 108B and the CPU 140 of the controller 125 in the operation system 104A receive the sent authenticating information, and determines whether or not the received authenticating information matches authenticating information stored in the HDDs 146 and 141 or determines whether or not the received authenticating information satisfies the authenticating condition.

When one of the pieces of the authenticating information does not match the stored information in the determination, in step S106, it is determined that the authenticating processing is not normally performed. Then, the controller 131 or 125 sets the refusal of a use (authentication fails). The information on the using refusal is sent to another controller, and the operation system 104A and the terminal device 108B are not used.

When the authenticating information matches the stored information in the determination in step S105, in step S107, the authenticating processing is normally performed and then set to the permission of a use (authentication is successful).

According to the fourth embodiment, when the authenticating processing is normally performed in step S107, in step S108, the controller 125 of the operation system 104A controls the operation for starting an application program for communication for the operation support with the controller 131 of the terminal device terminal device 108B. That is, the operation system 104A in the first operation room 103A for operation has an initiative so as to smoothly perform the operation by controlling the using operation for the operation support of the terminal device 108B on the support side.

By starting the application, the terminal device 108B is set to perform the operation support of the operation system 104A. That is, in step S109, an environment for smoothly performing the operation support is set.

When the supporter operates the keyboard 134 of the terminal device 108B from the first support room 109A and requests the transmission of the endoscope image to the operation system 104A in the first operation room 103A, the controller 125 of the operation system 104A determines that the supporter is the user whose request is permitted, it sends (distributes) the endoscope image.

When the supporter sends the information for operation support to the first operation room 103A, the controller 125 of the operation system 104A displays the sent information on the second monitor 129.

Thus, the operator in the first operation room 103A smoothly performs the operation by receiving the support information from the supporter in the first support room 109A.

FIG. 14 schematically shows the processing shown in FIG. 13.

On the operation system 104A side, the authenticating magnetic card 130b for authenticating the user is attached, thus performing the user authenticating processing of the operation system 104A. If the user is authenticated, the operator is authenticated by the operation system 104A.

The operator attaches the magnetic card 130a for inputting the patient information, thus inputting the patient information to the operation system 104A. The operation system 104A sets the normal operating state while the operation is not supported.

On the terminal device 108B side, the magnetic card 137a for authenticating the user is attached, thus normally performing the user authenticating processing of the terminal device 108B. Then, the terminal device 108B authenticates the user.

In order to set the using state for enabling the operation support, the operator attaches the magnetic card 130c for authenticating the target device (for operation support). The supporter attaches the magnetic card 137b for authenticating the target system, thus sending the information necessary for authentication to the partner.

For example, the operation system 104A sends the patient ID and the operator ID in addition to the IP address of the terminal device 108B to the terminal device 108B. The terminal device 108B sends the supporter ID and the patient ID in addition to the IP address of the operation system 104A to the operation system 104A. In this case, the authenticating information added to the IP address and sent may be encrypted by encrypting means (not shown) and may be sent.

The operation system 104A and the terminal device 108B to which the authenticating information is sent authenticate and determine whether or not the sent information matches the authenticating information registered therein (or whether or not the sent information satisfies the authenticating condition).

When it is determined that the information matches each other, the authenticating processing is normally performed, thus setting the using state. In the using state, at least the terminal device 108B is in an original connecting state in which the terminal device 108B does not take part in the operation control on the operation system 104A side.

The controller 125 of the operation system 104A sends the signal for controlling the application start operation to the controller 131 of the terminal device 108B. In accordance with the signal, the terminal device 108B starts the application (program) for supporting the operation.

The program starts and the supporter sends an operating command such as a request for sending the image from the terminal device 108B. Then, the operating command is received by the operation system 104A and is normally recognized as the operating command.

Further, the supporter sends support information for supporting the operation from the terminal device 108B.

According to the fourth embodiment, only when the authenticating processing is normally performed on both the operation system 104A and the terminal device 108B, the using state for supporting the operation is set and thus the leakage of information on the operation is highly prevented.

The operation system 104A sends the signal for starting the application for supporting the operation. Further, the operation system 104A starts the application which receives the operation support from the terminal device 108B. In this case, in the program for the same control operation as that for the control operation of the operation system 104A in a state in which the operation is not supported, preferably, the application having the expanded function is started in view of the improvement in operability by sending the image information to the terminal device 108B via the communication line 106 and by displaying the information received via the communication line 106 on the second monitor 129.

Alternatively, in a state in which the application having a function for communicating data with the terminal device 108B via the communication line 106 is limited in the function for communicating the data via the communication line 106, the application program starts and is used. When the operation is supported for the terminal device 108B after the authenticating processing to the terminal device 108B, the limit on the communication function may be reset. In this case, the preferable operability is ensured.

As mentioned above, the operator of the first operation room 103A smoothly performs the operation by receiving the support information from the supporter in the first support room 109A.

Upon sending the image information or support information, the ID information on the sender may be added and be sent together with the IP addresses on the sending destination and the sender, the ID information may be checked on the recipient. When the ID information are different from each other, the receiving may be stopped.

When the image information and the support information can be received and be sent between the operation system 104A and the terminal device 108B, the authentication may be performed at the proper time interval.

When the controller 125 of the operation system 104A receives the command for requesting the sending of the information different from the sent information, the controller 125 determines whether or not the information for the command is sent depending on the authenticating condition and the permission or refusal of the sending of the information may be performed depending on the determining result.

In the above description, the communication device 105A in the operation system 104A and the terminal device 108B in the first support room 109A are subjected to the authenticating processing so as to be used. Further, the communication device 105A in the operation system 104A and the terminal device 108A in the conference room 107 may be set to be used.

In this case, the supporter in the conference room 107 wants to monitor the endoscope image connected to the controller 125 in the first operation room 103A or the image by switching the CCU 123 for outputting the electrocardiogram image or the image output device such as the electrocardiogram measurement device 114.

In this case, it is convenient if the switching operation is controlled from the conference room 107 without troublesome operation of the operator. Therefore, according to the fourth embodiment, referring to FIG. 14, a command of a switching instructing signal is sent to the operation system 104A from the terminal device (108A in this case), thereby performing the corresponding switching operation.

That is, the controller 125 in the operation system 104A receives the switching instructing signal. Then, upon authenticating the supporter of the terminal device 108A, the controller 125 determines whether or not the set authenticating condition is satisfied. When it is determined that the set authenticating condition is satisfied, the controller 125 switches the control operation from a state in which the endoscope image is sent to a state in which the electrocardiogram image is sent and thus operability is improved. That is, the controller 125 controls the switching operation of the image output device for sending the image data in accordance with the operating instruction from the terminal device 108A based on the information in the authentication.

According to the fourth embodiment, as mentioned above, the leakage of information on the privacy is prevented by automatically performing the authenticating processing and, the operation is supported and the easy setting of the smooth operation is enabled. Further, the operator has the initiative and sets the state for the operation support, thus ensuring the preferable operability.

In the above description, the authenticating processing for the operation system 104A may be automatically performed by attaching, to the magnetic card reader 127, one magnetic card to which the ID information of the operator and the information for authenticating the terminal device for the operation support are written as well as the information for starting the operation system 104A. The terminal device 108B has the magnetic card having the functions of the magnetic cards 137a and 137b, and the magnetic card is attached. Thus, the authenticating processing of the operation system 104A may be performed.

### Fifth Embodiment

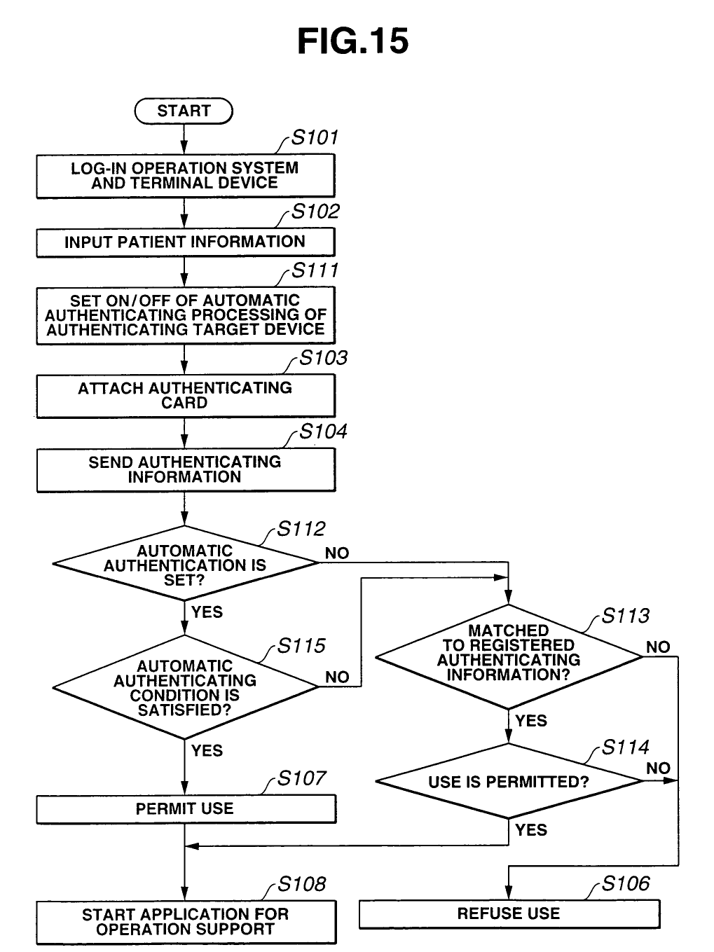

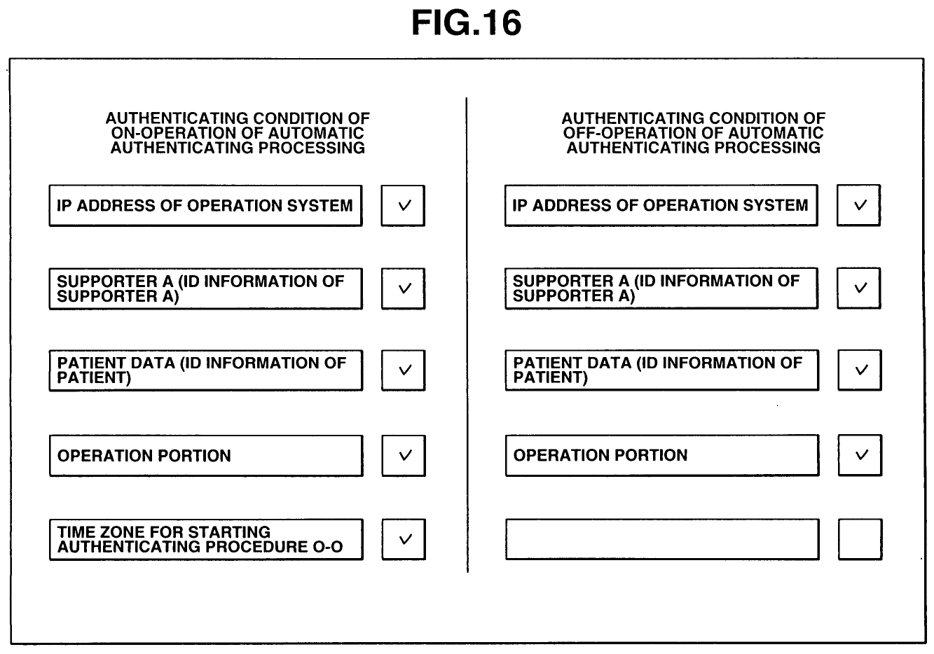

Next, a description is given of the fifth embodiment with reference to FIGS. 15 and 16. According to the fifth embodiment, the authenticating processing for the authenticated target device according to the fourth embodiment is set by selecting the automatic operation or the manual operation (or checking).

The structure of hardware according to the fifth embodiment is the same as that according to the fourth embodiment. However, a control program stored in the HDD 140 according to the fifth embodiment is different from that shown in FIG. 10 according to the fourth embodiment.

FIG. 15 shows a typical operation according to the fifth embodiment. In the operation shown in FIG. 15, in step S111, the on/off setting processing for the authenticating target device is performed, between steps S102 and S103 in FIG. 13 according to the fourth embodiment.

FIG. 16 shows one example for a setting screen in this case. On the setting screen shown in FIG. 16, the authenticating condition in the on-operation of the automatic authenticating processing additionally has the time zone for starting the authenticating procedure as well as the authenticating condition in the off-operation thereof (e.g., the IP address of the operation system, the supporter A (ID information on the supporter A), the patient data (e.g., the ID information of the patient), and the operation part).

The time zone for starting the authenticating procedure in this case is set to the time zone around or of a scheduled start time of the operation. Thus, when the authenticating procedure is performed at the time zone except for the operation time zone, the authenticating processing is not automatically performed and the security performance is improved.

According to the fifth embodiment, when the condition in the on-operation of the automatic authenticating processing is not satisfied, the authentication and determination are performed under the authenticating condition in the off-operation. Considering the case in which the authenticating procedure does not start at the time zone for the automatic authentication, it is designed that the authentication is accepted even under the non-automatic authenticating condition.

When the authenticating condition in the on-operation of the automatic authenticating processing is the same as the authenticating condition in the off-operation of the automatic authenticating processing in the setting shown in FIG. 16, the authenticating processing and the determining processing are performed only by the selected authenticating condition. In this case, when the on-operation of the automatic authenticating processing is selected, the operation is the same as that according to the fourth embodiment.

On the other hand, in the off-operation of the authenticating processing, the authenticating processing is manually performed. Since the determining result of the authenticating processing is indicated, the operator determines whether the use should be permitted or not based on the result.

Referring to FIG. 15, after the setting processing in step S111, the processing passes through steps S103 and S104 in FIG. 13 and then advances to step S112. According to the fifth embodiment, the terminal device 108J performs the same operation according to the fourth embodiment.

In step S112, the CPU 40 of the controller 125 determines whether or not the automatic authentication is set. If it is determined that the automatic authentication is not set, the processing shifts to step S113. In step S113, the CPU 40 of the controller 125 determines whether or not the information matches the authenticating information which is registered in the case of the non-automatic authentication. When it is determined that the sent authenticating information does not match the registered one, similarly to step S106 in the case shown in FIG. 13, the use is refused.

While, when it is determined that the sent authenticating information matches the registered one, in step S114, the matching is displayed. Further, the operator determines whether the use based on the result is permitted or refused. When it is determined that the use is permitted, the processing advances to step S108 whereupon the application for the operation support starts as mentioned according to the fourth embodiment.

On the other hand, when the operation support is not necessary, the refusal of use is selected and the processing advances to step S106.

In the determination in step S112 the automatic authentication is set and then the processing advances to step S115 whereupon the CPU 40 of the controller 125 determines whether or not the sent authenticating information satisfies the condition for automatic authentication.

When the CPU 40 of the controller 125 determines that the condition for automatic authentication is satisfied, the processing advances to step S107. The processing subsequent to step S107 is the same as that according to the fourth embodiment. When the CPU 40 of the controller 125 determines that condition for automatic authentication is not satisfied, the processing advances to above-mentioned step S113.

According to the fifth embodiment, in the automatic authenticating processing, the authenticating condition is strictly set as compared with the case of checking the authenticating processing, thereby setting the using environment state of the operation support under the condition for preventing the leakage of information on the privacy without fail.

According to the fourth and fifth embodiments, the operation for the operation system 104A is supported by the connection to the terminal device 108B in the first support room 109A or the terminal device 108A in the conference room 107 via the communication line 106. Further, the operation for one operation system may be supported by the connection to a plurality of terminal devise via the communication line 106.

FIG. 17 shows one example of this. In the operation system 104A, the terminal device 108B in the first support room 109A and the terminal device 108D in a ward with a nurse having a bed on which the patient lies are subjected to the authenticating processing, and are set to be used. In this case, the operation system 104A can send the endoscope image and the information on the operation device to the terminal device 108B under the authenticating condition (with high initiative). The terminal device 108B can send the support information to the operation system 104A.

On the contrary, for the terminal device 108D which is operated by the nurse who performs the nursing work at the ward of the patient, the authenticating condition with low initiative is applied so as to smoothly perform the corresponding nursing work, grasp the operation process, and only send the endoscope image from the operation system 104A.

As mentioned above, the setting state for sending and receiving the information between the operation system 104A and the plural terminal devices 108B and 108D is set depending on the support contents of the supporter who uses the terminal device, thereby smoothly supporting the operation.

The authenticating magnetic card 130c is attached, thereby automatically performing the authenticating processing. Further, another card such as an 1C card may be used.

According to the fourth and fifth embodiments, as mentioned above, when the operator performs the operation by using the operation system in the operation room in the hospital, the support information of the remote supporter is easily set via the communication line, thereby smoothly performing the operation.

According to the conventional arts, the connection between the operation room and the support room via the communication line needs the troublesome authenticating procedure (authenticating processing) so as to prevent the leakage of information on the privacy such as the patient information. However, with the system according to the fourth and fifth embodiments, the remote operation support system is realized to automatically perform the authenticating processing and to improve the operability for smoothing the operation support. Further, with system according to the fourth and fifth embodiments, the remote operation support system is realized to ensure the security on the information on the privacy such as the operation information and to smoothly perform the operation support.

As mentioned above, the operation support system according to the first to fifth embodiments realizes the high security performance.

The present invention is not limited to the first to fifth embodiments and can be modified without departing from the essentials of the present invention.

1. An operation support system for connecting a first device arranged in an operation room to a second device arranged in an operation support room other than the operation room via a communication line and for enabling the communication to an external device via the communication line, the operation support system comprising:
a setting portion which registers identification information for permitting the connection to a connecting request destination requesting the connection via the communication line; and
a determining portion which determines whether the connection to the connection request destination is permitted or refused depending on the presence or absence of the identification information registered by the setting portion.

2. An operation support system according to claim 1, wherein the determining portion determines whether the connection is permitted or refused by switching a table for registering the identification information based on a result of authenticating an individual operator.

3. An operation support system according to claim 1, wherein the determining portion determines whether the connection is permitted or refused by switching a table for registering the identification information based on a result of authenticating an individual patient.

4. An operation support system according to claim 1, wherein the setting portion comprises a storing portion, and registers the identification information in the storing portion in a table format.

5. An operation support system according to claim 1, wherein the identification information is an IP address.

6. An operation support system according to claim 5, wherein the determining portion determines whether the connection is permitted or refused by switching a table for registering the IP address based on a result of authenticating an individual operator.

7. An operation support system according to claim 5, wherein the determining portion determines whether the connection is permitted or refused by switching a table for registering the IP address based on a result of authenticating an individual patient.

8. An operation support system according to claim 5, wherein the first device in the operation room and the second device in the operation support room are connected to an Intranet via the communication line by an IP network, and at least one of the first device in the operation room and the second device in the operation support room is connected to an Internet by the IP network.

9. An operation support system according to claim 1, further comprising:
a control portion which executes and controls the connection to the connecting request destination based on a determining result of the determining portion as to whether the connection to the connecting request destination is permitted or refused.

10. An operation support system according to claim 9, wherein when the determining portion determines that the connection to the connecting request destination is permitted, the control portion executes the connection to the connecting request destination to enable the transmission of an image signal of an endoscope image formed by an endoscope device arranged in the operation room, and when the determining portion determines that the connection to the connecting request destination is refused, the control portion refuses the connection to the connecting request destination to disable the transmission of the image signal of the endoscope image.

11. An operation support system according to claim 1, wherein the identification information includes a telephone number of a dial-up network.

12. An operation support system for communicating information on an operation via a communication line between a first device for performing processing for the operation arranged in an operation room for the operation and a second device arranged in a support room for supporting the operation, the operation support system comprising:
an authenticating information sending portion which sends authenticating information to the first device and the second device via the communication line; and
an authenticating and determining portion which performs the authentication and determination depending on determination as to whether or not the authenticating information received by the first device and the second device matches the registered registered-information upon sending the authenticating information by the authenticating information sending portion.

13. An operation support system according to claim 12, wherein the authenticating information sending portion sends the authenticating information in accordance with an operation for attaching a card for recording the authenticating information.

14. An operation support system according to claim 12, wherein the first device controls a communication operation for supporting the operation of the second device after the authenticating processing of the authenticating and determining portion.

15. An operation support system according to claim 12, wherein the first device starts a program for a communication operation for supporting the operation of the second device.

16. An operation support system according to claim 12, wherein the second device controls an operation for switching an image output device for inputting image information to a controller forming the first device based on the information in the authentication.

17. An operation support system according to claim 12, wherein the first device forms an endoscope system for the operation using an endoscope.

18. An operation support system according to claim 12, wherein both the first device and the second device perform the authentication and determination in the authenticating processing, and the authenticating processing is successful only when both results of the authentication and determination are successful.

19. An automatic authenticating processing method for operation support for automatic authenticating processing in a system that connects a first device arranged in an operation room for performing processing of the operation to a second device arranged in a support room for the operation support via a communication line, the automatic authenticating processing method for operation support comprising:
an authenticating-condition setting step of setting an authenticating condition;
a sending step of sending authenticating information; and
a determining step of determining whether or not the sent authenticating information satisfies the authenticating condition.

20. An automatic authenticating processing method for operation support according to claim 19, wherein the sending step and the determining step are executed in accordance with the attachment of an authenticating card for storing the authenticating information.

21. An automatic authenticating processing method for operation support according to claim 19, wherein when the sent authenticating information satisfies the authenticating condition in the determining step, a program in the support room for supporting the operation starts.
